(19) United States
(12) Patent Application Publication
(10) Pub. No.: US 2011/0045497 A1
(43) Pub. Date: Feb. 24, 2011

(54) NOVEL ACIDIC GLYCAN MARKERS OF HUMAN CELLS

(75) Inventors: Suvi Natunen, Vantaa (FI); Tero Satomaa, Helsinki (FI); Jari Natunen, Vantaa (FI); Leena Valmu, Helsinki (FI); Virve Pitkänen, Helsinki (FI)

Correspondence Address: BIRCH STEWART KOLASCH & BIRCH, PO BOX 747, FALLS CHURCH, VA, 22040-0747, US

(73) Assignees: SUOMEN PUNAINEN RISTI, VERIPALVELU, Helsinki (FI); GLYKOS FINLAND LTD., Helsinki (FI)

(21) Appl. No.: 12/530,389

(22) PCT Filed: Mar. 7, 2008
(86) PCT No.: PCT/FI08/50110

§ 371 Date: Dec. 8, 2009

(30) Foreign Application Priority Data
Mar. 8, 2007 (FI) 20070199

(51) Int. Cl. G01N 33/53; C12Q 1/02; C12N 5/071; C12N 5/0775

(52) U.S. Cl. 435/7.21; 435/29; 435/366

(57) ABSTRACT

The invention is directed to the analysis of novel acidic glycan markers of several types of human cells. The analysis is performed by mass spectrometry or specific binder molecules.

### BACKGROUND

The present invention is in a preferred embodiment directed to disialic acid epitopes, wherein two sialic acid residues are linked to each other in a terminal non-reducing end epitope such as “disialic acids” including NeuNAcα8NeuNAcα3Gal with different variants on glycolipid structures especially on ganglioseries ganglioside GD3, referred as “ganglio disialic acid” and in a preferred embodiment much less known and rare epitope linked to a protein and/or N-acetyllactosamine structures and referred to as“protein/LacNAc disialic acid”. These structures are chemically different and characterize the cells separately in different manner. The invention is preferably directed to the use of GD3 recognizing antibody in context of hematopoietic or mesenchymal stem cells and cell differentiated thereof, or use of the ganglioseries specific GD3 antibody together with the different antibody recognizing disialic acid epitope on protein and/or N-acetyllactosamine. Due to cell type specificity of glycosylation, the glycans identified on embryonal stem cells do not predict glycosylation of hematopoietic or mesenchymal stem cells.

In background there is different, branched, glycolipid epitope GD2, NeuNAcα8NeuNAcα3(GalNAcβ4)Gal glycolipid, which can be recognized on certain mesenchymal stem cell preparations. It is realized that this is a different non-reducing end structure and the present invention is especially directed to antibodies, which do not cross-react or have much lower reactivity with this structure.

### SUMMARY OF THE INVENTION

The present invention is directed to the method for analyzing human stem cells, preferrably human hematopoietic stem cell, embryonal stem cell, or mesenchymal stem cell and the differentiated cells derived thereof, by analyzing the amount of or presence of unusual disialylated epitopes, including terminal non-reducing end structures:
a) In a preferred embodiment NeuXαNeuX-epitopes, wherein X is Ac or Gc, preferably Ac, referred also as “disialic acid” epitope, more preferably NeuNAcα8NeuNAcα3Gal-epitopes and even more preferably the disialic acid epitope is presented on N-acetyllactosamine. The invention is especially directed to two subtypes of NeuNAcα8NeuNAcα3Gal-epitopes and reagents recognizing these:
   a1) NeuNAcα8NeuNAcα3Gal on a protein and/or N-acetyllactosamine epitope, referred as “protein/LacNAc disialic acid”. Detection of “protein/LacNAc disialic acid” is especially preferred in context of hematopoietic stem cells and cells differentiated thereof.
   a2) NeuNAcα8NeuNAcα3Gal on ganglioseries ganglioside GD3, referred as “ganglio-disialic acid”. “Ganglio-disialic acid” is especially preferred in context of mesenchymal stem cells, preferably of corb blood origin, and more preferably of cells differentiated into osteogenic or adipogenic direction thereof.
b) The invention is further directed to recognition of “non-linear disialylated” N-acetyllactosamines comprising one sialic acid on position 3 of Gal and another one on position 6 of GlcNAc, wherein the epitope is on NeuXα3Galβ3(NeuXα6)GlcNAc, wherein X is Ac or Gc.

In a preferred embodiment the invention is directed to the analysis of disialylated epitopes linked to lipids or proteins.

In a preferred embodiment the disialylated N-acetyllactosamine is linked to protein.

The analysis is performed by using mass spectrometry and/or specific binding agent recognizing the target glycan such as “protein/LacNAc disialic acid” and/or “ganglio-disialic acid”. It is realized that mass spectrometric profiling can reveal the unusual structures comprising disialylated structures independent of the exact structures and the quantitative amounts of the specific monosaccharide compositions are characteristic to certain stem cell classes and/or to cells differentiated thereof. The invention also revealed that the disialylated structure could be recognized by specific binder molecules recognizing terminal disialylated epitopes These included antibody S2-566 (Seikagaku), especially when the structure was recognized on a protein linked glycan.

A preferred type of N-glycan to be analyzed has a preferred N-monosaccharide composition according to the Formula C

### SₖHₙNₚF_{q}

wherein
k is integer from 2 to 5,
n is integer from 3 to 6,
p is integer from 3 to 5, and
q is integer being 0 or 1,
S is Neu5Ac and/or Neu5Gc, H is hexose selected from group D-Man or D-Gal, N is N-D-acetylhexosamine, preferably GlcNAc or GalNAc, more preferably GlcNAc, and F is L-fucose.

The method is in a preferred embodiment directed to N-glycans, wherein the N-glycan comprises one disialyted N-acetyllactosamine, preferably the N-glycan comprises one disialyted N-acetyllactosamine epitope according to the formula NeuAcαNeuAcαGalβ4GlcNAc.

The disialylated N-acetyllactosamine epitope is in a preferred embodiment disialic epitope comprising preferably NeuAcαNeuAcα3Galβ4GlcNAc or NeuAcαNeuAcα6Galβ4GlcNAc, even more preferably NeuAcα8NeuAcα3Galβ4GlcNAc.

### BRIEF DESCRIPTION OF THE DRAWINGS

FIG. 1. FACS staining results of CD34 positive and negative cells with different GD3 antibodies. Percentages of CD34+ and CD34− cells having positive staining with different anti-disialic acid antibodies is shown. Antibodies were VIN-IS-56 from Chemicon with product code MAB4308, MB3.6 from BD Pharmingen product code 554274, 4F6 from Covalab product code mab0014 and S2-566 from Seikagaku (product code 270554).

FIG. 2. FACS staining results of cord blood derived hematopoietic stem cells with a GD3 antibody. Percentage of CD34 and CD133 positive cells as well as CD34 and CD133 negative cells having positive staining with anti-GD3 S2-566 (Seikagaku product code 270554) is shown.

FIG. 3. FACS staining results of mesenchymal stem cells (MSC) and osteogenically differentiated (OG) as well as adipogenically differentiated (AG) cells with different GD3 antibodies. Mesenchymal stem cells were either derived from bone marrow (A) or from cord blood (B). Percentages of cells having positive staining with different anti-disialic acid antibodies are shown. Antibodies were VIN-IS-56 from Chemicon with product code MAB4308, MB3.6 from BD Pharmingen product code 554274, 4F6 from Covalab product code mab0014, S2-566 from Seikagaku product code 270554 and 4i283 from US Biological product code G2005-67.

FIG. 4. FACS analysis of mesenchymal stem cells (MSC) and osteogenically differentiated (OG) and adipogenically (AG) differentiating/differentiated cells from bone marrow (BM) and cord blood (CB) with antibody S2-566 (Seikagaku product code 270554).

FIG. 5. Stem cell nomenclature.

FIG. 6. Immunoblotting of hematopoietic stem cell lysate by anti-disialic acid antibody. Cell lysates of CD34+ and CD34− cells were blotted with S2-566 (Seikagaku product code 270554) and VIN-IS-56 (Chemicon product code MAB4308) and visualization of detected protein is shown.

### DESCRIPTION OF THE INVENTION

The present invention is directed to the method for analyzing human stem cells or cells differentiated thereof by analyzing the amount of or presence of unusual disialylated epitopes, including terminal non-reducing end structures:
a) In a preferred embodiment NeuXαNeuX-epitopes, wherein X is Ac or Gc, preferably Ac, referred also as “disialic acid” epitope, more preferably NeuNAcα8NeuNAcα3Gal-epitopes and even more preferably the disialic acid epitope is presented on N-acetyllactosamine. The invention is especially directed to two subtypes of NeuNAcα8NeuNAcα3Gal-epitopes and reagents recognizing these:
   a1) NeuNAcα8NeuNAcα3Gal on a protein and/or N-acetyllactosamine epitope, referred to as “protein/LacNAc disialic acid”. Detection of “protein/LacNAc disialic acid” is especially preferred in context of hematopoietic stem cells and cells differentiated thereof
   a2) NeuNAcα8NeuNAcα3Gal on ganglioseries ganglioside GD3, referred to as “ganglio disialic acid”. “Ganglio disialic acid” is especially preferred in context of mesenchymal stem cells, preferably of corb blood origin, and more preferably of cells differentiated into osteogenic or adipogenic direction thereof.
b) The invention is further directed to recognition of “non-linear disialylated” N-acetyllactosamines comprising one sialic acid on position 3 of Gal and another one on position 6 of GlcNAc, wherein the epitope is on NeuXα3Galβ3(NeuXα6)GlcNAc, wherein X is Ac or Gc.

In a preferred embodiment the invention is directed to the analysis of lipid or protein linked disialylated epitopes.

In a preferred embodiment the disialylated N-acetyllactosamine is linked to protein.

In a preferred embodiment the disialylated epitope is a) a N-glycan comprising at least two sialic acid residues per one N-acetyllactosamine, preferably comprising one disialylated N-acetyllactosamine unit, when sialic acid is NeuGc or NeuAc and N-acetyllactosamine is Galβ3/4GlcNAc, in a preferred embodiment associated with terminal non-reducing end disialylated structures disialic acid or non-linear disialylated N-acetyllactosamine. b) N-glycan type structures including an N-glycan or similar size oligosaccharide comprising two sialic acids on a core structure unusual to structure by a protein N-glycosidase enzyme cleaving normally linkage between asparigine and reducing end GlcNAc of N-glycan. This group comprises unusual epitopes, which comprise two sialic acid residues cleavable by a type sialidase enzyme mainly specific for α3-linked sialic acid indicating potential branches in structure with NeuXα3-terminals.

Disialic Acid Epitopes and Binders Recognizing these

Preferred lipid linked NeuXαNeuX-epitope includes NeuXα8NeuXα3Gal-epitopes on GD3 gangliosides: with structures NeuXα8NeuXα3Galβ4GlcβCer where X can be Ac or Gc. The GD3 ganglioside is especially preferred for the characterization of hematopoietic stem cells and/or mesenchymal stem cells and cells differentiated thereof. It was revealed that the protein and/or N-acetyllactosamine linked epitope NeuXα8NeuXα3Gal(βGlcNAc) characterizes the cells differently than the glycolipid epitope NeuXα8NeuXα3Galβ4GlcβCer comprising glucose residue at the core.

### Disialic Acid N-Acetyllactosamine Epitopes

The disialylated N-acetyllactosamine epitope is in a preferred embodiment disialic epitope comprising preferably NeuAcαNeuAcα3Galβ4GlcNAc or NeuAcαNeuAcα6Galβ4GlcNAc, even more preferably NeuAcα8NeuAcα3Galβ4GlcNAc.

The invention also revealed that the structure can be recognized by specific binder molecules recognizing terminal disialylated epitopes especially when the structure is recognized on N-acetyllactosamine such as NeuAcα8NeuAcα3GalβGlcNAc, preferably NeuAcα8NeuAcα3Galβ4GlcNAc, preferably on a protein. The preferred binders include antibody S2-566 (Seikagaku), especially when the structure is recognized on N-acetyllactosamine such as NeuAcα8NeuAcα3GalβGlcNAc, preferably NeuAcα8NeuAcα3Galβ4GlcNAc, preferably on a protein.

### Combination Use of Ganglio—and Protein/lacNAc Disialic Acid Binders

In a preferred embodiment the invention is directed to use of combination of specific disialic acid recognizing antibodies wherein the first antibody can recognize the protein and/or lactosamine linked epitope NeuXα8NeuXα3Gal(βGlcNAc), preferably NeuXα8NeuXα3Gal(βGlcNAc), preferably specifically or exclusively and the second antibody has specificity recognizing specifically or exclusively of the epitope NeuXα8NeuXα3Gal-on glycolipids, preferably on GD3, but not the protein and/or N-acetyllactosamine linked epitope.

### Exclusive and Dual Specificity Protein/lacNAc and Ganglio Disialic Acid Binding Antibodies

It is realized that the different epitopes can be observed between “protein/LacNAc disialic acid” and “ganglio disialic acid” binding antibodies. In a preferred embodiment the invention is directed to methods and binder reagents with exclusive specificity.

In a preferred embodiment the invention is directed to exclusively “ganglio disialic acid” specific binder, wherein the binder, such as an antibody, binds to “ganglio disialic acid”, but does not recognize the protein or N-acetyllactosamine linked epitope.

In a preferred embodiment the invention is directed to exclusively “protein/LacNAc disialic acid” specific binder, wherein the binder, such as an antibody, binds to “protein/lacNAc disialic acid”, but does not recognize the “ganglio disialic acid” epitope.

The invention is further directed to dual specificity antibody, wherein the antibody can recognize both the “ganglio disialic acid” epitope and “protein/N-acetyllactosamine disialic acid” epitope.

Analysis by Specific Binders and/or Mass Spectrometry

The analysis is preferably performed by using mass spectrometry and/or by using specific glycan binding agent. It is realized that mass spectrometric profiling can reveal the unusual structures comprising disialylated structures independent of the exact structures and the quantitative amounts of the specific monosaccharide compositions are characteristic to the cells.

### Preferred N-Glycan Structures

A preferred type of N-glycan to be analyzed has a preferred N-monosaccharide composition according to the Formula C

### SₖHₙNₚF_{q}

wherein
k is integer from 2 to 5,
n is integer from 3 to 6,
p is integer from 3 to 5, and
q is integer being 0 or 1,
S is Neu5Ac and/or Neu5Gc, H is hexose selected from group D-Man or D-Gal, N is N-D-acetylhexosamine, preferably GlcNAc or GalNAc, more preferably GlcNAc, and F is L-fucose.

The method is in a preferred embodiment directed to N-glycans, wherein the N-glycan comprises one disialyted N-acetyllactosamine, preferably the N-glycan comprises one disialyted N-acetyllactosamine epitope according to the formula NeuAcαNeuAcαGalβ4GlcNAc. The preferred binders for the structure includes, antibody S2-566 (Seikagaku) and antibodies with similar specificity.

The preferred structure of the N-glycan is according to Formula OS1

(NeuAcα)ₘGalβ(Fucα3/4)ₙ₁GlcNAcβ2Manα3([Manα6]ₙ₂)Manβ4GlcNAcβ4(Fucα6)ₙ₃GlcNAc,
wherein n1, n2 and n3 integers 0 or 1, with the provision, that when n1 is 0 then n3 is 1 and when n1 is 1 then n3 is 0 or both n1 and n3 are 0 and wherein m is integer 2 or 3.

More preferably the structure of the N-glycan is according to the Formula

(NeuAcα)ₘGalβGlcNAcβ2Manα3([Manα6]ₙ₂)Manβ4GlcNAcβ4(Fucα6)ₙ₃GlcNAc,
wherein the variables are as described for formula OS1, and even more preferably NeuAcαNeuAcαGalβGlcNAcβ2Manα3([Manα6]ₙ₂)Manβ4GlcNAcβ4(Fucα6)ₙ₃GlcNAc, wherein the variables are as described for formula OS1.

### Disialyted N-Acetyllactosamine Epitope NeuXα3Galβ3(NeuXα6)GlcNAc

In a separate embodiment the N-glycan comprises one disialyted N-acetyllactosamine epitope according to the formula NeuXα3Galβ3(NeuXα6)GlcNAc, wherein X is Ac or Gc, and preferably the N-glycan has composition S2G1H5N4 and S1G2H5N4.

More preferably the structure of the N-glycan is according to the Formula

(NeuXα)ₘ₁GalβGlcNAcβ2Manα3([NeuXα]ₘ₂GalβGlcNAcβ2Manα6)Manβ4GlcNAcβ4GlcNAc,
wherein X is either Gc or Ac, with the prevision that there is at least one Gc or Ac in the molecule and that there can be both Gc and Ac in disialic acid epitopes and m1 is 2 and m2 is 1, or m2 is 2 and m1 is 1, and sialic acid residues are either α3- or α6-linked to Gal or a6-linked to GlcNAc or α8- or α9-linked to each other. The Gal residues are either β3 and/or β4 linked.

More preferably the structure of the N-glycan is according to the Formula

### NeuXαGalβ3(NeuXα6)GlcNAcβ2Manα3 (NeuXαGalβ3GlcNAcβ2Manα6)Manβ4GlcNAcβ4Glc NAc

and/or other branch isomer

NeuXαGalβ3GlcNAcβ2Manα3 (NeuXαGalβ3(NeuXα6)GlcNAcβ2Manα6)Manβ4GlcNAcβ4Glc NAc.

Disialylated Glycan with Composition S2H4N5F1

The invention is further directed to the disialylated glycan, which has composition S2H4N5F1, preferably the glycan has structure according to the formula

GlcNAcβ{(NeuAcα)₂Galβ3GlcNAcβ2Manα3(GlcNAcβ2Manα6)[GlcNAcβ]Manβ4GlcNAcβ4(Fu cα6)GlcNAc},
or

### (NeuAcα)₂GalβGlcNAcβ2Manα3 (GlcNAcβ2Manα6)[GlcNAcβ4]Manβ4GlcNAcβ4(Fucα6)GlcN Ac

or

### (NeuAcα)₂GalβGlcNAcβ2Manα3 (GalNAcβGlcNAcβ2Manα6)Manβ4GlcNAcβ4(Fucα6)GlcNAc

or
NeuAcαGalβGlcNAcβ2Manα3 (NeuAcαGalNAcGlcNAcβ2Manα6)Manβ4GlcNAcβ4(Fucα6)Gl cNAc.

### Preferred Cell Types

The preferred cells to be analyzed includes stem cells and cells differentiated from these. The preferred stem cell is selected from the group of human hematopoietic stem cell, embryonic stem cell or mesenchymal stem cell and preferred cells are directly derived thereof. Preferably the hematopoietic cells and mesenchymal stem cells are cord blood or bone marrow derived human cells.

### Analysis of Cell Status

The invention is especially directed to analysis of the status of the stem cells preferably including

a) differentiation status of cells and/or
b) differences in cell types, in preferred embodiment the analysis of the differentiation may be analysed between stem cell and cell type differentiated from the stem cell including both differentiation status and cell type status analysis, and/or
c) contamination status preferably with regard to effect of exogenous carbohydrate materials such as antigenic or immunogenic carbohydrates from cell culture or purification reagents.

In a specific embodiment the invention is directed to analysis of contamination in stem cell preparation by analysis of disialyated structures, preferably including the analysis of characteristic disialylated epitopes and analysis of presence of unusual sialic acids, preferably NeuGc in the disialylated epitopes. Here the word “contamination” also includes the risk of contamination by exogenous material and analysis of contamination includes also analysis of risk of contamination by exogenous materials such as cell culture materials aimed for the use with the cells according to the invention.

In a preferred embodiment the NeuGc contamination is analyzed from cells which have been in contact with exogenous carbohydrate materials, such as non-human materials preferably animal (referring here to non-human animals) material, such as animal cells (including feeder cells) or animal material derived cell culture materials such as glycoproteins, monosaccharides, oligosaccharides, glycans or glycolipids. In a preferred embodiment protein associated contamination is analyzed, including analysis of glycoproteins of the cells according to the invention and/or the glycoproteins of the exogenous material. In a separately preferred embodiment glycolipid associated contamination is analyzed, including analysis of glycolipids of the cells according to the invention and/or the glycolipids of the exogenous material.

In a preferred embodiment the differention and/or cell type status and the contamination status of the cells are analyzed.

### Novel Oligosialylated N-Glycans Comprising at Least One N-Acetyllactosamine Residue

The present invention revealed novel oligosialylated N-glycan structures from stem cells and corresponding differentiated cells comprising at least two sialic acid residues per N-acetylactosamine or one disialylated N-acetyllactosamine unit. In a preferred embodiment the glycans are monoantennary glycans comprising only one N-acetyllactosamine residue. The stem cells are preferably human stem cells.

The invention revealed that the one N-acetyllactosamine and two sialyl-residue comprising glycans are useful for characterization of multiple types of stem cells and their derivatives including hematopoietic, embryonal and mesenchymal stem cells.

### Preferred Terminal (NeuAcα)₂GalβGlcNAc Epitopes

The (NeuAcα)₂GalβGlcNAc, LacNAc disialic acid, epitope correspond to two types of terminal structures,

1) disialyl-structures, the sialic acids are linked to each other preferably by α8- and/or α9-linkages, more preferably α8-linkages and
2) non-linear disialylalted-structures, wherein the sialic acids are linked to Gal and GlcNAc in type 1 N-acetyllactosamine structure: NeuXα3Galβ3(NeuXα6)GlcNAc, wherein X is Gc and/or Ac.

### Preferred Terminal NeuAcαNeuAcαGalβ4GlcNAc-Epitopes

It is realized that type two N-acetyllactosamine, observable e.g. by specific β4-galactosidase (e.g. S. pneumoniae galactosidase) digestions, is a major glycan type in N-glycans of embryonal stem cells and therefore invention is more preferably directed to terminal epitope structures corresponding to the oligosialyl epitope of especially in S2/3H3N3/4F0/1-structures (e.g. Formula OS3 below): NeuAcαNeuAcα3/6Galβ4GlcNAc, NeuAcα8NeuAcα3/6Galβ4GlcNAc, even more preferably NeuAcα8NeuAcα3Galβ4GlcNAc.

These structures are especially preferred for the S2H3N3/4F1-structures found from embryonal stem cells and for homologous S2/3H3N4F0/1-structures of hematopoietic cells, S2H3N3/4F0/1-structures found from mesenchymal type cells and furthermore terminal GlcNAc comprising S2H4N5F1-structures of embryonal stem cells.

### Preferred Terminal NeuXα3Galβ3(NeuXα6)GlcNAc-Epitopes

In a preferred embodiment the invention is directed to type 1 N-acetyllactosamine structure: NeuXα3Galβ3(NeuXα6)GlcNAc, wherein X is Gc and/or Ac, preferably at least one of the X groups being Gc, in context of NeuGc comprising biantennary glycans especially the NeuGc-comprising N-glycans of embryonal stem cells.

### Monosaccharide Compositions and Mass Spectrometric Signals

A preferred group of the oligosialylated structures in the glycomes of stem cells are the glycans corresponding to following mass spectrometric signals, the preferred monosaccharide compositions are given after the signals:

signal at m/z 1694 (S2H3N3)
signal at m/z 1840 (S2H3N3F1),
signal at m/z 1856 (S2H4N3),
signal at m/z 2002 (S2H4N3F1),
signal at m/z 2294 (S3H4N3F1),
signal at m/z 2408 is (S2H4N5F1),
signal at m/z 2528 is (S2G1H5N4), and
signal at m/z 2544 is (S1G2H5N4).
S is Neu5Ac, G is Neu5Gc, H is hexose selected from group D-Man or D-Gal, N is N-D-acetylhexosamine, preferably GlcNAc or GalNAc, more preferably GlcNAc, and F is L-fucose.

The preferred monosaccharide compositions are thus according to the Formula C

### SₖHₙNₚF_{q}

### Wherein

k is integer from 2 to 5,
n is integer from 3 to 6,
p is integer from 3 to 5, and
q is integer being 0 or 1,
S is Neu5Ac and/or Neu5Gc, H is hexose selected from group D-Man or D-Gal, N is N-D-acetylhexosamine, preferably GlcNAc or GalNAc, more preferably GlcNAc, and F is L-fucose.

The signals are given for deprotonated singly charged ions for negative ion mode analysis e.g. by MALDI-TOF mass spectrometry and it is obvious for person skilled in the art that based on the monosaccharide compositions several other signals corresponding to the same molecular compositions can be measured such as other analyzable non-covalent adduct ions (such as potassium and/sodium adduct) or signals or compositions corresponding monosaccharide compositions of the glycans or chemical derivatives of the glycans

### The Preferred Group of S2/3H3N3/4F0/1-Structures

A preferred group of the oligosialylated structures in the glycomes of stem cells are the glycans corresponding to

signal at m/z 1694 (S2H3N3)
signal at m/z 1840 (S2H3N3F1),
signal at m/z 1856 (S2H4N3), and
signal at m/z 2002 (S2H4N3F1) and
signal at m/z 2294 (S3H4N3F1).

It is realized this group comprises similar monosaccharide compositions. The glycans have similarity in composition with the oligosialylated structures present in embryonal stem cells in hematopoietic stem cells and in mesenchymal stem cells. Thus this type of structures are preferred for methods, especially analysis, directed to multiple types stem cells. Most preferably the invention is directed to the recognition of human stem cells.

In a preferred embodiment the invention is directed to analysis of structure of preferred oligosialylated N-glycans with compositions S2H3N3, S2H3N3F1, S2H4N3, S2H4N3F1, and S3H4N3F1, when the composition comprises monoantennary N-glycan type structures according to the

### Formula OS1

(NeuAcα)ₘGalβ(Fucα3/4)ₙ₁GlcNAcβ2Manα3([Manα6]ₙ₂)Manβ4GlcNAcβ4(Fucα6)ₙ₃GlcNAc,

Wherein n1, n2 and n3 integers 0 or 1, with the pro vision, that when n1 is 0 then n3 is 1 and when n1 is 1 then n3 is 0 or both n1 and n3 are 0

and
wherein m is integer 2 or 3.

More preferably the composition comprise the structures according to the Formula (NeuAcα)ₘGalβGlcNAcβ2Manα3([Manα6]ₙ₂)Manβ4GlcNAcβ4(Fucα6)ₙ₃GlcNAc, wherein the variables are as described for formula OS1.

### Analysis Methods by Mass Spectrometry or Specific Binding Reagents

The invention is specifically directed to the recognition of the terminal structures by either specific binder reagents and/or by mass spectrometric profiling of the glycan structures.

In a preferred embodiment the invention is directed to the recognition of the structures and/or compositions based on mass spectrometric signals corresponding to the structures.

The preferred binder reagents are directed to characteristic epitopes of the structures such as terminal epitopes and or characteristic branching epitopes, such as monoantennary structures comprising a Manα-branch or not comprising a Manα-branch.

In another preferred embodiment the invention is directed to the recognition of the terminal oligosialic acid epitopes comprising a N-acetyllactosamine and at least two sialic acid residues. The preferred binder is antibody, more preferably a monoclonal antibody.

In a preferred embodiment the invention is directed to a monoclonal antibody specifically recognizing at least one of the structures selected form the group NeuXα3Galβ3(NeuXα6)GlcNAc, more preferably NeuAcα3Galβ3(NeuGcα6)GlcNAc, and/or NeuGcα3Galβ3(NeuAcα6)GlcNAc. In a separate embodiment antibody binds to NeuXα3Galβ3(NeuXα6)GlcNAc and binds effectively essentially independent of presence of NeuGc in the structure, it is realized that such antibody would effectively recognized several isomeric forms of the structure and thus be effective in recognition of preferred structures.

In a preferred embodiment the invention is directed to a monoclonal antibody specifically recognizing at least one of the structures selected form the group NeuAcαNeuAcα3/6Galβ3GlcNAc, more preferably NeuAcα8NeuAcα3Galβ4GlcNAc, and/or NeuAcα8NeuAca6Galβ4GlcNAc. In a preferred embodiment the invention is directed to the use of the antibody when it recognizes NeuAcα8NeuAcα3Galβ4GlcNAc or shorter epitope NeuAcα8NeuAcαGal, it is realized that numerous such antibodies and methods for using these are known in the art.

### Oligosialylated Lactosamine Structures in N-Glycomes of CD133+ Hematopoietic Stem Cells

The invention reveals novel oligosialylated structures present in hematopoitic stem cells. MALDI TOF mass spectrometry in negative ion mode revels signals at m/z 1856 and m/z 2294. The signals indicate glycan structures specifically present in cord blood derived CD133 positive hematopoietic stem cells but not in corresponding CD 133 negative hematopoietic stem cells, see Table 1.

The invention is in a preferred embodiment directed to use of the mass spectrometric signals for analysis of hematopoietic stem cells.

Preferred monosaccharide composition assigned for signal at m/z 1856 is S2H4N3, and m/z 2294 is S3H4N3F1.

The preferred S2/3H3N4F0/1-Structures

A preferred subgroups of the oligosialylated structures in the glycomes of hematopoietic stem cells are the glycans corresponding to

signal at m/z 1856 (S2H4N3), and
signal at m/z 2294 (S3H4N3F1). These form a group of preferred similar compositions. The glycans have similarity in composition with the oligosialylated structures present in embryonal stem cells and in mesenchymal stem cells. Thus this type of structures is preferred for methods, especially analysis, directed to multiple types stem cells.

In a preferred embodiment the invention is directed to analysis of structure of preferred oligosialylated N-glycans with compositions S2H3N3 and S2H4N3F1, when the composition comprises monoantennary N-glycan type structures

### Formula OS2

(NeuAcα)ₘGalβ(Fucα3/4)ₙ₁GlcNAcβ2Manα3(Manα6)Manβ4GlcNAcβ4(Fucα6)ₙ₃GlcNAc,

Wherein n1, and n3 are integers 0 or 1, with the pro vision, that when n1 is 0 then 32 is 1 and

when n1 is 1 then n3 is 0; and
wherein m is integer 2 or 3.

More preferably the composition comprise the structures according to the Formula (NeuAcα)₂GalβGlcNAcβ2Manα3(Manα6)Manβ4GlcNAcβ4(Fucα6)ₙ₃GlcNAc, wherein the variables are as described for formula 0S3.

### Human Embryonic Stem Cells

The invention is directed to novel oligosialylated structures present in embryonal stem cells. MALDI TOF mass spectrometry in negative ion mode showed signals at m/z 1840 and m/z 2002, m/z 2408, m/z 2528, and m/z 2544. The signals indicate glycan structures specifically present in embryonal stem cells at certain differentiation stages, but not present or more weakly present in control cells (mEF), see Table 2. The invention is in preferred embodiment directed to the use of the specific signals for the analysis of embryonal type stem cells at various stages of differentiation.

The preferred monosaccharide composition assigned for the signal at m/z 1840 is S2H3N3F1, for the signal at m/z 2002 is S2H4N3F1, for the signal at m/z 2408 is S2H4N5F1, for the signal at m/z 2528 is S2G1H5N4, and for the signal at m/z 2544 is S1G2H5N4. The invention is directed to oligosaccharides and oligosaccharide derivatives, especially glycosidically modified and/or permethylated oligosaccharide compositions for the analysis of embryonal stem cells.

The invention is in preferred embodiment directed to the use glycan structures with the preferred monosaccharide compositions for the analysis of embryonal type stem cells at various stages of differentiation

### Preferred Subgroup of S2H3N3/4F1-Structures

A preferred subgroups of the oligosialylated structures in the glycomes of embryonal stem cells are the glycans corresponding to signal at m/z 1840 (S2H3N3F1), and to the signal at m/z 2002 (S2H4N3F1) with similar compositions. The glycans have similarity in composition with the oligosialylated structures present in hematopoietic stem cells and oligosialylated structures in mesenchymal stem cells. Thus this type of structures is preferred for methods, especially analysis, directed to multiple types of embryonal stem cells.

In a preferred embodiment the invention is directed to analysis of structure of preferred oligosialylated N-glycans with compositions S2H3N3F1 and S2H3N4F1, when the composition comprises monoantennary N-glycan type structures Formula OS3

(NeuAcα)₂Galβ(Fucα3/4)ₙ₁GlcNAcβ2Manα3([Manα6]ₙ₂)Manβ4GlcNAcβ4(Fucα6)ₙ₃GlcNAc,

Wherein n1, n2 and 3 integers 0 or 1, with the pro vision, that when n1 is 0 then n2 is 1 and

when n1 is 1 then n2 is 0.

More preferably the composition comprise the structures according to the Formula (NeuAcα)₂GalβGlcNAcβ2Manα3([Manα6]ₙ₂)Manβ4GlcNAcβ4(Fucα6)ₙ₃GlcNAc,

wherein the variables are as described for formula OS3.

### Preferred Subgroup of S2H4N5F1-Structures

Preferred subgroups of the oligosialylated structures in the glycomes of embryonal stem cells are the glycans corresponding to signal at m/z 2408 (S2H4N5F1). The glycans have special decrease in amount during the differentiation of the embryonal stem cells as shown in Table 2. Thus this type of structures is preferred for methods, especially analysis methods, directed to stem cells, in a preferred embodiment to embryonal type stem cells.

### Terminal GlcNAc Comprising S2H4N5F1-Structures

In a preferred embodiment the invention is directed to analysis of structure of preferred oligosialylated N-glycans with compositions S2H4N5F1 structures, when the composition comprises biantennary N-glycan type structures with terminal diasialyl-epitope and terminal HexNAc structures, which are in a preferred embodiment GlcNAc residues. The GlcNAc residues correspond preferably to GlcNAcβ2 and an additional branching GlcNAc linked to N-glycan core such as in terminal HexNAc-structures, in a preferred embodiment linked to Manβ4-structure:

GlcNAcβ{(NeuAcα)₂GalβGlcNAcβ2Manα3(GlcNAcβ2Manα6)[GlcNAcβ]Manβ4GlcNAcβ4(Fu cα6)GlcNAc}, and more preferably

### (NeuAcα)₂GalβGlcNAcβ2Manα3(GlcNAcβ2Manα6)[GlcNAcβ4]Manβ4GlcNAcβ4(Fucα6)GlcN Ac

### LacdiNAc Comprising S2H4N5F1-Structures

In a preferred embodiment the invention is directed to analysis of structure of preferred oligosialylated N-glycans with compositions S2H4N5F1 structures, when the composition comprises biantennary N-glycan type structures with terminal LacdiNAc structure. The lacdiNAc epitope has structure GalNAcβ3GlcNAc, preferably GalNAcβ4GlcNAc and preferred sialylated LacdiNAc epitope has the structure NeuAcα6GalNAcβ34GlcNAc, based on the known mammalian glycan structure information. The preferred sialyl-lactosamine structures includes NeuAcα3/6Galβ3GlcNAc.

The invention is especially directed to the composition with terminal diasialyl-epitope and terminal LacdiNAc structure according to the Formula

### (NeuAcα)₂GalβGlcNAcβ2Manα3(GalNAcβ3GlcNAcβ2Manα6)Manβ4GlcNAcβ4(Fucα6)GlcNAc

and/or
terminal sialyl-lactosamine epitope and a sialylated LacdiNAc epitope according to the Formula NeuAcαGalβ3GlcNAcβ2Manα3(NeuAcαGalNAcGlcNAcβ2Manα6)Manβ4GlcNAcβ4(Fucα6)Gl cNAc.

It is realized that the sialyl-LacdiNac comprising structure does not comprise necessarily terminal disialyl epitope, but the glycan is classified to this group as an unusual two sialic acid comprising glycan, which is further associated with the differentiation of embryonal stem cells.

### Preferred Subgroup of S2G1H5N4 and S1G2H5N4 Comprising Structures

In a preferred embodiment the invention is directed to analysis of structure of preferred oligosialylated N-glycans with compositions S2G1H5N4 and S1G2H5N4 structures, when the composition comprises two N-acetyllactosamines and three sialic acid residues, which are preferably either NeuGc (G) or NeuAc (S) residues, and thus at least two sialic acid residues per N-acetyllactosamine unit.

This structure group is especially preferred in context of embryonal stem cells. It further realized that it is useful to analyze the NeuGc comprising structures in context of contamination by animal protein. In another preferred embodiment the composition is analyzed in context of contamination by animal protein recognizing the terminal disialic acid epitope of the glycans. In a specifically preferred embodiment the terminal epitope is type I N-acetyllactosamine disialoepitope NeuXα3Galβ3(NeuXα6)GlcNAc similar to potential contaminating animal protein.

The invention is preferably directed to the structures according to the Formula OS-Gc

(NeuXα)ₘ₁GalβGlcNAcβ2Manα3([NeuXα]ₘ₂GlcNAcβ2Manα6)Manβ4GlcNAcβ4GlcNAc,
wherein X is either Gc or Ac, with the prevision that there is at least one Gc or Ac in the molecule and that there can be both Gc and Ac in disialic acid epitopes and m1 is 2 and m2 is 1, or
m2 is 2 and m1 is 1, and sialic acid residues are either α3- or α6-linked to Gal or a6-linked to GlcNAc or α8- or α9-linked to each other. The Gal residues are either β3 and/or β4 linked.

In a preferred embodiment the structures according to the Formula OS-Gc comprise type II N-acetyllactosamine and two sialic acid residues

(NeuXα)ₘ₁Galβ4GlcNAcβ2Manα3([NeuXα]ₘ₂Galβ4GlcNAcβ2Manα6)Manβ4GlcNAcβ4GlcN Ac
more preferably

### NeuXαNeuXαGalβ4GlcNAcβ2Manα3(NeuXαGalβ4GlcNAcβ2Manα6)Manβ4GlcNAcβ4GlcN Ac

and/or other branch isomer

### NeuXαGalβ3GlcNAcβ2Manα3(NeuXαNeuXαGalβ4GlcNAcβ2Manα6)Manβ4GlcNAcβ4GlcN Ac

In a separate preferred embodiment the structures according to the Formula OS-Gc comprise type I N-acetyllactosamine and two sialic acid residues

NeuXαGalβ3(NeuXα6)GlcNAcβ2Manα3(NeuXαGalβ3GlcNAcβ2Manα6)Manβ4GlcNAcβ4Glc NAc

and/or other branch isomer

### NeuXαGalβ3GlcNAcβ2Manα3(NeuXαGalβ3(NeuXα6)GlcNAcβ2Manα6)Manβ4GlcNAcβ4Glc NAc.

### Mesenchymal Stem Cells

The invention is directed to novel oligosialylated structures present in mesenchymal stem cells and cell differentiated from mesenchymal stem cells, referred to together as mesenchymal type stem cells.

MALDI TOF mass spectrometry in negative ion mode showed signals at m/z 1694, at m/z 1840, at m/z 1856, and at m/z 2002. The signals indicate glycan structures specifically present in mesenchymal type cells at certain differentiation stages, but not present in cell culture media controls (Abserum indicating human AB-blood group serum, or FCS indicating fetal calf serum), see Table 3. The invention is in preferred embodiment directed to the use of the specific signals for the analysis of mesenchymal type cells stem cells at various stages of differentiation.

### The Preferred S2H3N3/4F0/1-Structures

A preferred group of the oligosialylated structures in the glycomes of mesenchymal stem cells are the glycans corresponding to

signal at m/z 1694 (S2H3N3)
signal at m/z 1840 (S2H3N3F1),
signal at m/z 1856 (S2H4N3), and
signal at m/z 2002 (S2H4N3F1).

It is realized this group comprises similar monosaccharide compositions. The glycans have similarity in composition with the oligosialylated structures present in embryonal stem cells and oligosialyted structures in hematopoietic stem cells. Thus this type of structures are preferred for methods, especially analysis, directed to multiple types stem cells, in a preferred embodiment to mesenchymal type stem cells.

In a preferred embodiment the invention is directed to analysis of structure of preferred oligosialylated N-glycans with compositions S2H3N3, S2H3N3F1, S2H4N3 and S2H4N3F1, when the composition comprises monoantennary N-glycan type structures according to the Formula OS4

(NeuAcα)₂Galβ(Fucα3/4)ₙ₁GlcNAcβ2Manα3([Manα6]₂)Manβ4GlcNAcβ4(Fucα6)ₙ₃GlcNAc,

Wherein n1, n2 and n3 integers 0 or 1, with the pro vision, that when n1 is 0 then n3 is 1 and

when n1 is 1 then n3 is 0 or both n1 and n3 are 0.

More preferably the composition comprise the structures according to the Formula (NeuAcα)₂GalβGlcNAcβ2Manα3([Manα6]ₙ₂)Manβ4GlcNAcβ4(Fucα6)ₙ₃GlcNAc,

wherein the variables are as described for formula OS4.

### Unusual Disialyl—and Other Sialyl-Structure Compositions

The invention is further directed to the preferred disialylepitopes according to the invention independent of the core structure. The invention is especially directed to the analysis of stem cell glycan structures, especially embryonal stem cell glycans, wherein these comprise unusual glycan structures with composition S2H2N3F1, mass spectrometric signal m/z 1679 in negative mode; and S2H4N2F1, signal at m/z 1800. The signals were increased during differentiation The invention is further directed to specific analysis of presence of mass signal and/or monosaccharide compositions of unusual glycans with compositions S1H6N4F1Ac, S1H7N5F1Ac, the invention is preferably directed to the specific structures when the structures comprise the sialic acid modified by O-Acetyl group, preferably selected from the group 7,8, or 9-O-acetyl group on NeuAc, most preferably 9-OAc. The invention is especially directed to the recognition of the sialic acid, when it is in structures Ac-NeuAcα3/6Galβ3GlcNAc, the sialic can be recognized as mass spectrometric fragment in mass spectrometric scan or by monoclonal antibody recognizing the epitope, preferably linked to N-glycan. The invention is especially directed to the glycans and analysis of the acetylated sialic acid in context of differentiation embryonal stem cells to stage 2 or stage 3 cells.

### Stem Cell Nomenclature

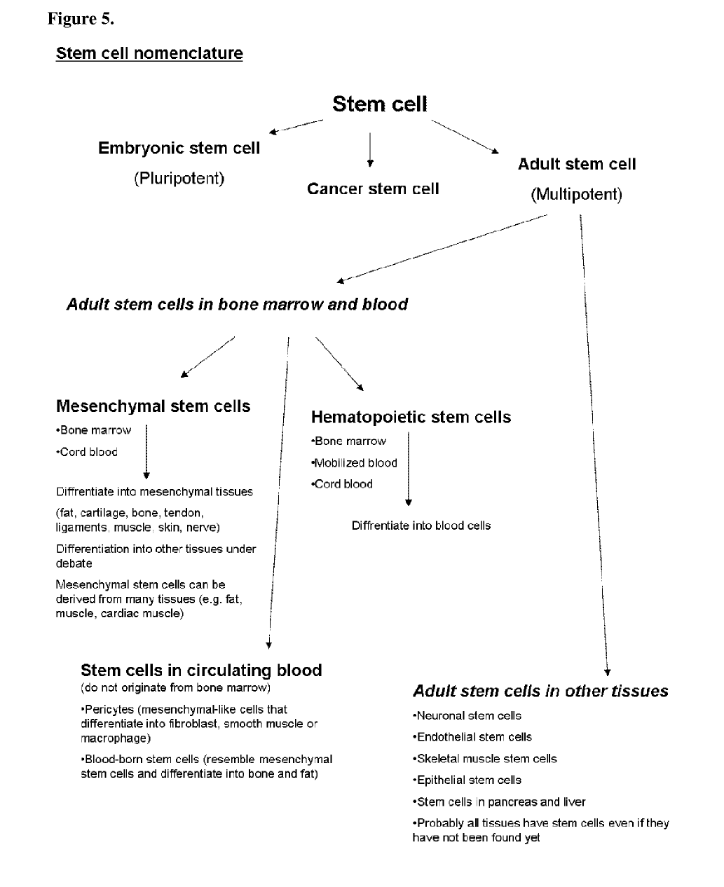

The present invention is directed to analysis of all stem cell types, preferably human stem cells. A general nomenclature of the stem cells is described in FIG. 5. The alternative nomenclature of the present invention describe early human cells which are in a preferred embodiment equivalent of adult stem cells (including cord blood type materials) as shown in FIG. 5. Adult stem cells in bone marrow and blood are equivalent for stem cells from “blood related tissues”.

### Preferred Types of Early Human Cells

The invention is directed to specific types of stem cells also referred as early human cells based on the tissue origin of the cells and/or their differentiation status.

The present invention is specifically directed to early human cell populations meaning multipotent cells and cell populations derived thereof based on origins of the cells including the age of donor individual and tissue type from which the cells are derived, including preferred cord blood as well as bone marrow from older individuals or adults.

Preferred differentiation status based classification includes preferably “solid tissue progenitor” cells, more preferably “mesenchymal-stem cells”, or cells differentiating to solid tissues or capable of differentiating to cells of either ectodermal, mesodermal, or endodermal, more preferentially to mesenchymal stem cells.

The invention is further directed to classification of the early human cells based on the status with regard to cell culture and to two major types of cell material. The present invention is preferably directed to two major cell material types of early human cells including fresh, frozen and cultured cells.

### Cord Blood Cells, Embryonal-Type Cells and Bone Marrow Cells

The present invention is specifically directed to early human cell populations meaning multipotent cells and cell populations derived thereof based on the origin of the cells including the age of donor individual and tissue type from which the cells are derived.
a) from early age-cells such 1) as neonatal human, directed preferably to cord blood and related material, and 2) embryonal cell-type material
b) from stem and progenitor cells from older individuals (non-neonatal, preferably adult), preferably derived from human “blood related tissues” comprising, preferably bone marrow cells.

### Cells Differentiating to Solid Tissues, Preferably to Mesenchymal Stem Cells

The invention is specifically under a preferred embodiment directed to cells, which are capable of differentiating to non-hematopoietic tissues, referred as “solid tissue progenitors”, meaning to cells differentiating to cells other than blood cells. More preferably the cell population produced for differentiation to solid tissue are “mesenchymal-type cells”, which are multipotent cells capable of effectively differentiating to cells of mesodermal origin, more preferably mesenchymal stem cells.

Most of the prior art is directed to hematopoietic cells with characteristics quite different from the mesenchymal-type cells and mesenchymal stem cells according to the invention.

Preferred solid tissue progenitors according to the invention includes selected multipotent cell populations of cord blood, mesenchymal stem cells cultured from cord blood, mesenchymal stem cells cultured/obtained from bone marrow and embryonal-type cells. In a more specific embodiment the preferred solid tissue progenitor cells are mesenchymal stem cells, more preferably “blood related mesenchymal cells”, even more preferably mesenchymal stem cells derived from bone marrow or cord blood.

Under a specific embodiment CD34+ cells as a more hematopoietic stem cell type of cord blood or CD34+ cells in general are excluded from the solid tissue progenitor cells.

### Fresh and Cultured Cells

### Fresh Cells

The invention is especially directed to fresh cells from healthy individuals, preferably non-modulated cells, and non-manipulated cells.

The invention is in a preferred embodiment directed to “fresh cells” meaning cells isolated from donor and not cultivated in a cell culture. It is realized by the invention that the current cell culture procedures change the status of the cells. The invention is specifically directed to analysis of fresh cell population because the fresh cells corresponding closely to the actual status of the individual donor with regard to the cell material and potential fresh cell population are useful for direct transplantation therapy or are potential raw material for production of further cell materials.

The inventors were able to show differences in the preferred fresh cell populations derived from early human cells, most preferably from cord blood cells. The inventors were able to produce especially “homogeneous cell populations” from human cord blood, which are especially preferred with various aspects of present invention. The invention is further directed to specific aspects of present invention with regard to cell purification processes for fresh cells, especially analysis of potential contaminations and analysis thereof during the purification of cells.

In a more preferred embodiment the fresh cells are materials related to/derived from healthy individuals. The healthy individual means that the person is not under treatment of cancer, because such treatment would effectively change the status of the cells, in another preferred embodiment the healthy person is receiving treatment of any other major disease including other conditions which would change the status of the cells.

It is realized that in some cases fresh cells may be needed to be produced for example for cell transplantation to a cancer patient using cells previously harvested from such a patient, under a separate embodiment the present invention is further directed to analysis of and other aspects of invention with regard to such cell material.

### Non-Modulated Cells

Even more preferably the fresh cells are “non-modulated cells” meaning that the cells have not been modulated in vivo by treatments affecting growth factor or cytokine release. For example stem cells may be released to peripheral blood by growth factors such as CSF (colony stimulating growth factor). Such treatment is considered to alter the status of cells from preferred fresh cells. The modulation may cause permanent changes in all or part of the cells, especially by causing differentiation.

### Non-Manipulated Cells

Even more preferably the fresh cells are “non-manipulated cells” meaning that the cells have not been manipulated by treatments permanently altering the status of the cells, the permanent manipulation including alterations of the genetic structure of the cells. The manipulations include gene transfection, viral transduction and induction of mutations for example by radiation or by chemicals affecting the genetic structures of the cells.

### Limited Fresh Cells Excluding Certain Specifically Selected Hematopoietic Stem Cell Populations

A more preferred limited group of fresh cells is directed to especially to effectively solid tissue forming cells and their precursors. Under specific embodiment this group does not include specifically selected more hematopoietic stem cell like cell populations such as
a) cell population selected as CD34+ cells from peripheral blood or bone marrow and
b) in another limited embodiment also total bone marrow and peripheral blood mononuclear cells are excluded.

It is realized that the fresh cell populations may comprise in part same cells as CD34+ when the cells are not selected with regard to that marker. It is realized that the exact cell population selected with regard to the marker are not preferred according to the invention as solid tissue forming cells.

Another limited embodiment excludes specifically selected CD34+ cell populations from cord blood and/or total mononuclear cells from cord blood. The invention is further directed to limited fresh cell populations when all CD34+ cell populations and/or all total cell populations of peripheral blood, bone marrow and cord blood are excluded. The invention is further directed to the limited fresh cell populations when CD34+ cell population were excluded, and when both CD34+ cell populations and all the three total cell populations mentioned above are excluded.

### Cultured Cells

The inventors found specific glycan structures in early human cells, and preferred subpopulations thereof according to the invention when the cells are cultured. Certain specific structures according to the invention were revealed especially for cultured cells, and special alterations of the specific glycans according to the invention were revealed in cultured cell populations.

The invention revealed special cell culture related reagents, methods and analytics that can be used when there is risk for by potentially harmful carbohydrate contaminations during the cell culture process.

### Cultured Modulated Cells

It is further realized that the cultured cells may be modulated in order to enhance cell proliferation. Under specific embodiment the present invention is directed to the analysis and other aspects of the invention for cultured “modulated cells”, meaning cells that are modulated by the action of cytokines and/or growth factors. The inventors note that part of the early changes in cultured cells are related to certain extent to the modulation.

The present invention is preferably directed to cultured cells, when these are non-manipulated. The invention is further directed to observation of changes induced by manipulation in cell populations especially when these are non-intentionally induced by environmental factors, such as environmental radiation and potential harmful metabolites accumulating to cell preparations.

### Preferred Types of Cultured Cells

The present invention is specifically directed to cultured solid tissue progenitors as preferred cultured cells. More preferably the present invention is directed to mesenchymal-type cells and embryonal-type cells as preferred cell types for cultivation. Even more preferred mesenchymal-type cells are mesenchymal stem cells, more preferably mesenchymal stem cells derived from cord blood or bone marrow.

Under separate embodiment the invention is further directed to cultured hematopoietic stem cells as a preferred group of cultured cells.

### Subgroup of Multipotent Cultured Cells

The present invention is especially directed to cultured multipotent cells and cell populations. The preferred multipotent cultured cell means various multipotent cell populations enriched in cell cultures. The inventors were able to reveal special characteristics of the stem cell type cell populations grown artificially. The multipotent cells according to the invention are preferably human stem cells.

### Cultured Mesenchymal Stem Cells

The present invention is especially directed to mesenchymal stem cells. The most preferred types of mesenchymal stem cells are derived from blood related tissues, referred as “blood-related mesenchymal cells”, most preferably human blood or blood forming tissue, most preferably from human cord blood or human bone marrow or in a separate embodiment are derived from embryonal type cells. Mesenchymal stem cells derived from cord blood and from bone marrow are preferred separately.

### Cultured Embryonal-Type Cells and Cell Populations

The inventors were able to reveal specific glycosylation nature of cultured embryonal-type cells according to the invention. The present invention is specifically directed to various embryonal type cells as preferred cultivated cells with regard to the present invention.

### Early Blood Cell Populations and Corresponding Mesenchymal Stem Cells

### Cord Blood

The early blood cell populations include blood cell materials enriched with multipotent cells. The preferred early blood cell populations include peripheral blood cells enriched with regard to multipotent cells, bone marrow blood cells, and cord blood cells. In a preferred embodiment the present invention is directed to mesenchymal stem cells derived from early blood or early blood derived cell populations, preferably to the analysis of the cell populations.

### Bone Marrow

Another separately preferred group of early blood cells is bone marrow blood cells. These cell do also comprise multipotent cells. In a preferred embodiment the present invention is directed to mesenchymal stem cells derived from bone marrow cell populations, preferably to the analysis of the cell populations.

### Preferred Subpopulations of Early Human Blood Cells

The present invention is specifically directed to subpopulations of early human cells. In a preferred embodiment the subpopulations are produced by selection by an antibody and in another embodiment by cell culture favouring a specific cell type. In a preferred embodiment the cells are produced by an antibody selection method preferably from early blood cells. Preferably the early human blood cells are cord blood cells.

The CD34 positive cell population is relatively large and heterogenous. It is not optimal for several applications aiming to produce specific cell products. The present invention is preferably directed to specifically selected non-CD34 populations meaning cells not selected for binding to the CD34-marker, called homogenous cell populations. The homogenous cell populations may be of smaller size mononuclear cell populations for example with size corresponding to CD 133+ cell populations and being smaller than specifically selected CD34+ cell populations. It is further realized that preferred homogenous subpopulations of early human cells may be larger than CD34+ cell populations.

The homogenous cell population may a subpopulation of CD34+ cell population, in preferred embodiment it is specifically a CD133+ cell population or CD133-type cell population. The “CD133-type cell populations” according to the invention are similar to the CD133+ cell populations, but preferably selected with regard to another marker than CD133. The marker is preferably a CD133-coexpressed marker. In a preferred embodiment the invention is directed to CD133+ cell population or CD133+ subpopulation as CD133-type cell populations. It is realized that the preferred homogeneous cell populations further includes other cell populations than which can be defined as special CD133-type cells.

Preferably the homogenous cell populations are selected by binding a specific binder to a cell surface marker of the cell population. In a preferred embodiment the homogenous cells are selected by a cell surface marker having lower correlation with CD34-marker and higher correlation with CD133 on cell surfaces. Preferred cell surface markers include a3-sialylated structures according to the present invention enriched in CD133-type cells. Pure, preferably complete, CD133+ cell population are preferred for the analysis according to the present invention.

The present invention is in a preferred embodiment directed to native cells, meaning non-genetically modified cells. Genetic modifications are known to alter cells and background from modified cells. The present invention further directed in a preferred embodiment to fresh non-cultivated cells.

The invention is directed to use of the markers for analysis of cells of special differentiation capacity, the cells being preferably human blood cells or more preferably human cord blood cells.

### General Method for Isolation of Cells or Cellular Components Comprising the Target Structures.

The invention is directed to process of isolation cell or cell component fraction involving the contacting the binder molecule epitope according to the invention. Corresponding target structures are expressed on stem cells and can be used to isolate the enriched target structure containing cell populations.

The preferred method to isolate cellular component includes following steps

1) Providing a stem cell sample.
2) Contacting the binder molecule according to the invention with the corresponding target structures on the cells or cell fractions.
3) Isolating the complex of the binder and target structure from at least from part of the cells or cellular materials.

Preferred methods for isolation of cells includes selection by immunomagnetic beads or by other cell sorting means in a preferred embodiment by FACS.

The isolation of cellular components according to the invention means production of a molecular fraction comprising increased (or enriched) amount of the glycans comprising the target structures according to the invention in method comprising the step of binding of the binder molecule according to the invention to the corresponding target structures, which are glycan structures bound by the specific binder.

It is realized that the components are in general enriched in specific fractions of cellular structures such as cellular membrane fractions including plasma membrane and organelle fractions and soluble glycan comprising fractions such as soluble protein, lipid or free glycans fractions. It is realized that the binder can be used to total cellular fractions.

In a preferred embodiment the target structures are enriched within a fraction of cellular proteins such as cell surface proteins releasable by protease or detergent soluble membrane proteins.

Use of the Binding Reagents for the Analysis of Cells and/or Cellular Interactions

It is realized that the carbohydrate structures on cell surfaces are associated with contacts with other cells and surrounding cellular matrix. Therefore the identified cell surface glycan structures and especially binding reagents specifically recognizing these are useful for the analysis of the cells.

The preferred analysis method includes the step of contacting the cell with a binding reagent and evaluating the effect of the binding reagent to the cell. In a preferred embodiment the cells are contacted with the binder under cell culture condition. In a preferred embodiment the binder is represented in multivalent or more preferably polyvalent form or in another preferred embodiment in surface attached form. The effect may be change in the growth characteristics or cellular signalling in the cells.

### FACS and Antibody Data

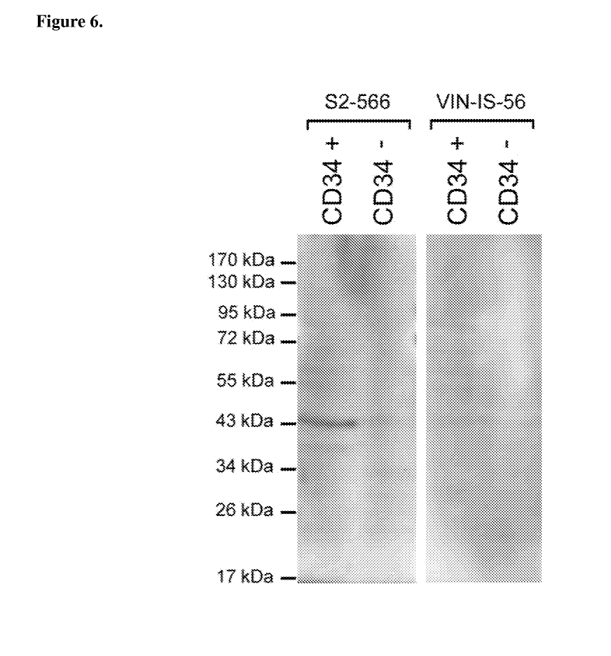

FACS data revealed that the anti-disialic acid antibody with protein/N-acetyllactosaminen specificity labeled effectively major part of CD34+ hematopoietic stem cells and even more effectively CD133+. The invention is in a preferred embodiment directed to the disialic acid epitope carrying protein as a marker for hematopoietic stem cells. The data further revealed a single protein labeled specifically in the CD34+ cells with “protein/LacNAc disialic acid” antibody. The invention is especially directed to a protein and/or its disialylated glycan epitope as marker for hematopoietic stem cells, see FIG. 6.

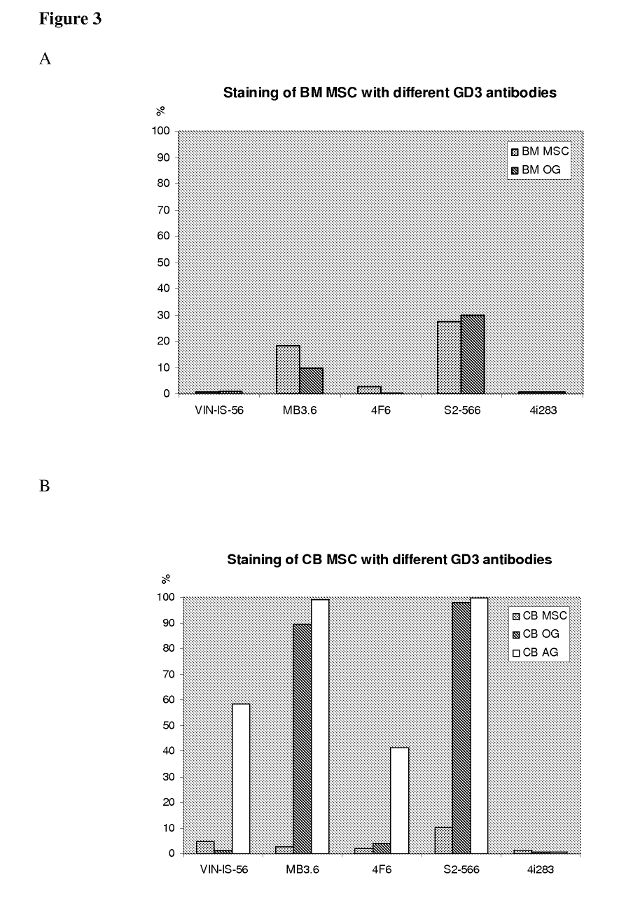

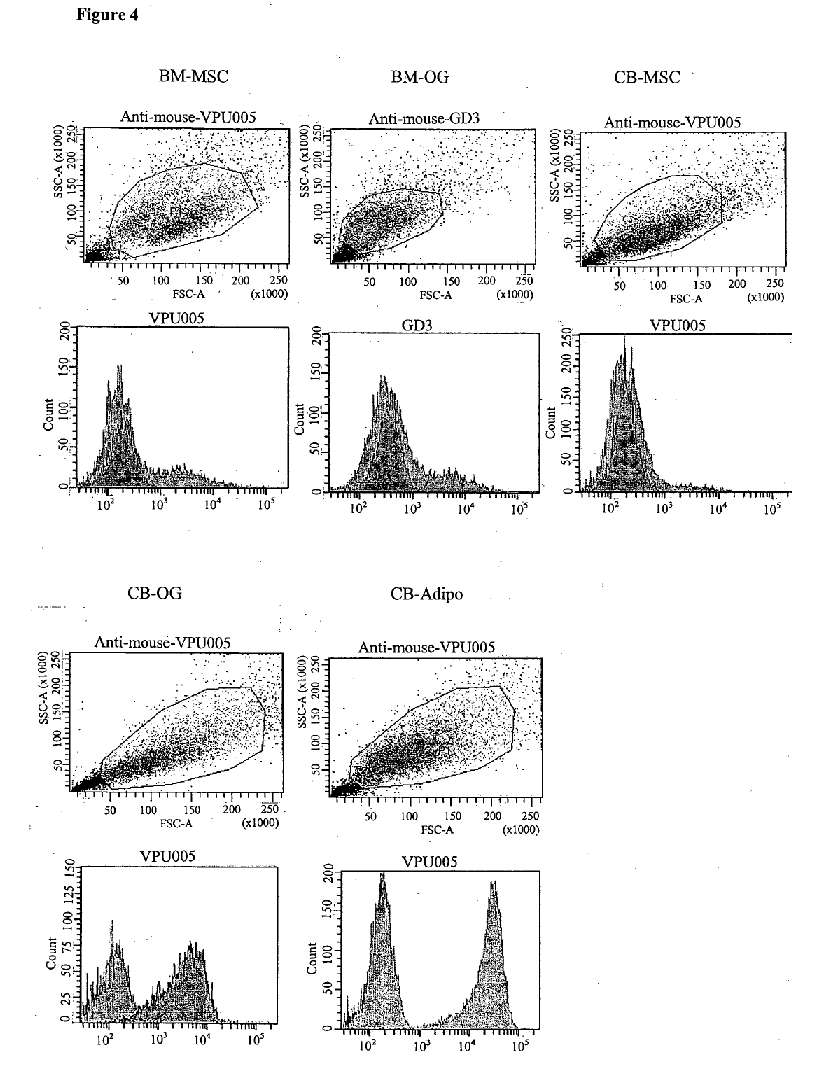

Fluorescence activated cell sorting (FACS) was used for analysis of mesenchymal cells, preferably of cord blood origin. Here FACS analysis revealed minor population of positive cells in mesenchymal stem cells and increasing amounts in osteogenically differentiated cells and typically even higher amounts in adipocyte differentiated cells, FIGS. 3 and 4. The invention is especially directed to the recognition of the cells based on the relative amounts of cells with specific labeling level of the antibodies, as exemplified by labeling patterns shown in the FACS analysis. The invention is especially directed to continuously changing target antigen amounts in osteoblactic (OB) cells and novel completely labeled cells as shown in for adipocytiyte (AC) differentiated cells, and cell populations with similar FACS patterns especially when labeled with equivalent of the antibodies used. The invention is further directed to the separation of the specific cell population, which is labeled, by positive selection and non-labeled cells by negative selection by the antibodies, and optionally further separating a partially reactive cell population. The invention is further directed to method of characterization of the specific mesenchymal cell populations, wherein the cell is labelled with the antibodies, preferably anti-disialic epitope antibody or antibodies, according to the invention and preferably the population has FACS profile essentially according to FIG. 4.

The invention is further directed to the specific isolated cell populations, preferably essentially similar to population binding to diasialyl epitope specific antibodies, preferably for characterization and/or therapeutic development of the cell population. The invention is especially directed to hematopietic stem cells or differentiated mesenchymal cells and cell population, wherein the cells are labeled with binder for disialylated specific epitope, preferably non-reducing end terminal epitope specific antibody.

Recognition of Structures from Glycome Materials and on Cell Surfaces by Binding Methods

The present invention revealed that beside the physicochemical analysis by mass spectrometry several methods are useful for the analysis of the structures. The invention is especially directed to a method:
i) Recognition by molecules binding glycans referred as the binders.
These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity).

a) Native glycosyltransferases (Rauvala et al.(1983) PNAS (USA) 3991-3995) and glycosidases (Rauvala and Hakomori (1981) J. Cell Biol. 88, 149-159) have lectin activities.
b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3).
c) Natural lectins, which are structurally homologous to glycosidases are also known indicating the continuity of the genus enzymes and lectins (Sun, Y-J. et al. J. Biol. Chem. (2001) 276 (20) 17507-14).

The genus of the antibodies as carbohydrate binding proteins without enzymatic activity is also very close to the concept of lectins, but antibodies are usually not classified as lectins.

Obviousness of the Peptide Concept and Continuity with the Carbohydrate Binding Protein Concept

It is further realized that proteins consist of peptide chains and thus the recognition of carbohydrates by peptides is obvious. E.g. it is known in the art that peptides derived from active sites of carbohydrate binding proteins can recognize carbohydrates (e.g. Geng J-G. et al (1992) J. Biol. Chem. 19846-53).

As described above antibody fragment are included in description and genetically engineed variants of the binding proteins. The obvious geneticall engineered variants would included truncated or fragment peptides of the enzymes, antibodies and lectins.

Useful binder specifies including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Neatherlands and internet databases such as pubmed/espacenet or antibody databases such as www.glyco.is.ritsumei.ac.jp/epitope/, which list monoclonal antibody glycan specificities.

### Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

Antibodies. Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corγnebacterium parvum.

A monoclonal antibody to a peptide motif(s) may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-I 1, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

### Methods Involving the Binder Molecules

### Recognition of Glycans of Mesenchymal Cells

General observations. The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificities in order to find high specificity antibodies.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells. It is realized that tissues comprising stem cells usually contain these in primitive stem cell stage and highly expressed markers according can be optimised or selected for the cell isolation. It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificties in order to find high specificity antibodies. Furthermore the invention is directed to the search of the structures from phage display libraries.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells.

It is realized that tissues comprising stem cells usually contain these in primitive stem cell stage and highly expressed markers according can be optimised or selected for the cell isolation. In a preferred embodiment the invention is directed to selection of mesenchymal cells by the binders according to the invention such as asialoganglioside recognizing proteins including preferably monoclonal antibodies recognizing the glycan epitopes according the invention. In a separate embodiments the invention is directed to the use of lectins or lectin homologous proteins optimized for the recognition.

It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

### Combinations

Combination of several antibodies for specific analysis of a population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%), are selected for the analytic method in combination with at least one “non-binding antibody”, recognizing preferably minor part (preferably from detection limit of the method to low level of recognition, in order of preference less than 10%, 7%, 5%, 2% or 1% of cells, e.g 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is directed to the use of several reagents recognizing terminal epitopes together, preferably at least two reagents, more preferably at least three epitopes, even more preferably at least four, even more preferably at least five, even more preferably at least six, even more preferably at least seven, and most preferably at least 8 to recognize enough positive and negative targets together. It is realized that with high specificity binders selectively and specifically recognizing elongated epitopes, less binders may be needed e.g. these would be preferably used as combinations of at least two reagents, more preferably at least three epitopes, even more preferably at least four, even more preferably at least five, most preferably at least six antibodies. The high specificity binders selectively and specifically recognizing elongated epitopes binds one of the elongated epitopes at least inorder of increasing preference, 5, 10, 20, 50, or 100 fold affinity, methods for measuring the antibody binding affinities are well known in the art. The invention is also directed to the use of lower specificity antibodies capable of effective recognition of one elongated epitope but also at least one, preferably only one additional elongated epitope with same terminal structure

The reagents are preferably used in arrays comprising in order of increasing preference 5, 10, 20, 40 or 70 or all reagents shown in cell labelling experiments.

The antibodies recognize certain glycan epitopes revealed as target structures according to the invention. It is realized that specificities and affinities of the antibodies vary between the clones. It was realized that certain clones known to recognize certain glycan structure does not necessarily recognize the same cell population.

Release of Binders or Binder Conjugates from the Cells by Carbohydrate Inhibition

The invention is in a preferred embodiment directed to the release of glycans from binders. This is preferred for several methods including:
a) release of cells from soluble binders after enrichment or isolation of cells by a method involving a binder
b) release from solid phase bound binders after enrichment or isolation of cells or during cell cultivation e.g. for passaging of the cells

The inhibiting carbohydrate is selected to correspond to the binding epitope of the lectin or part(s) thereof. The preferred carbohydrates includes oligosaccharides, monosaccharides and conjugates thereof. The preferred concentrations of carbohydrates includes contrations tolerable by the cells from 1 mM to 500 mM, more preferably 10 mM to 250 mM and even more preferably 10-100 mM, higher concentrations are preferred for monosaccharides and method involving solid phase bound binders.

Examples of monovalent inhibition condition are shown in Venable A. et al. (2005) BMC Developmental biology, for inhibition when the cells are bound to polyvalently to solid phase larger epitopes and/or concentrations or multi/polyvalent conjugates are preferred. The invention is further directed to methods of release of binders by protease digestion similarly as known for release of cells from CD34+ magnetic beads.

### Immobilized Binders

The present invention is directed to the use of the specific binder for or in context of cultivation of the stem cells wherein the binder is immobilized.

The immobilization includes non-covalent immobilization and covalent bond including immobilization method and further site specific immobilization and unspecific immobilization.

A preferred non-covalent immobilization methods includes passive adsorption methods. In a preferred method a surface such as plastic surface of a cell culture dish or well is passively absorbed with the binder. The preferred method includes absorption of the binder protein in a solvent or humid condition to the surface, preferably evenly on the surface. The preferred even distribution is produced using slight shaking during the absorption period preferably form 10 min to 3 days, more preferably from 1 hour to 1 day, and most preferably over night for about 8 to 20 hours. The washing steps of the immobilization are preferably performed gently with slow liquid flow to avoid detachment of the lectin.

### Specific Immobilization

The specific immobilization aims for immobilization from protein regions which does not disturb the binding of the binding site of the binder to its ligand glycand such as the specific cell surface glycans of stem cells according to the invention. Preferred specific immobilization methods includes chemical conjugation from specific aminoacid residues from the surface of the binder protein/peptide. In a preferred method specific amino acid residue such as cysteine is cloned to the site of immobilization and the conjugation is performed from the cystein, in another preferred method N-terminal cytsteine is oxidized by periodic acid and conjugated to aldehyde reactive reagents such as amino-oxy-methyl hydroxylamine or hydrazine structures, further preferred chemistries includes “click” chemistry marketed by Invitrogen and aminoacid specific coupling reagents marketed by Pierce and Molecular probes.

A preferred specific immobilization occurs from protein linked carbohydrate such as O- or N-glycan of the binder, preferably when the glycan is not close to the binding site or longer specar is used.

### Glycan Immobilized Binder Protein

Preferred glycan immobilization occurs through a reactive chemoselective ligation group R1 of the glycans, wherein the chemical group can be specifically conjugated to second chemoselective ligation group R2 without major or binding destructive changes to the protein part of the binder. Chemoselective groups reacting with aldehydes and ketones includes as amino-oxy-methyl hydroxylamine or hydrazine structures. A preferred R1-group is a carbonyl such as an aldehyde or a ketone chemically synthesized on the surface of the protein. Other preferred chemoselective groups includes maleimide and thiol; and “Click”-reagents including azide and reactive group to it.

Preferred synthesis steps includes
a) chemical oxidation by carbohydrate selectively oxidizing chemical, preferably by periodic acid, or
b) enzymatic oxidation by non-reducing end terminal monosaccharide oxidizing enzyme such as galactose oxidase or by transferring a modified monosaccharide residue to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute.

Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

### Conjugates Including High Specificity Chemical Tag

In a preferred embodiment the binder is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen and specific antibody for the peptide/antigen

### Preferred Conjugate Structures

The preferred conjugate structures are according to the

### Formula CONJ

B-(G-)ₘR1-R2-(S1-)ₙT-,
wherein B is the binder, G is glycan (when the binder is glycan conjugated), R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.

### Complex of Binder

The invention id further directed to complexes in of the binders involving conjugation to surface including solid phase or a matrix including polymers and like. It is realized that it is especially useful to conjugate the binder from the glycan because preventing cross binding of binders or effects of the binders to cells.

A complex comprising structure according to the

### Formula COMP

B-(G-)ₘR1-R2-(S1-)ₙ(T-)ₚ(L-)ᵣ₋(S2)ₛ-SOL,
wherein B is the binder, SOL is solid phase or matrix or surface or Label (may be also Ligand conjugated label), G is glycan (when the binder is glycan conjugated), R1 and R2 are chemoselective ligation groups, T is tag, preferably biotin, L is specifically binding ligand for the tag; S1 and S2 are optional spacer groups, preferably C₁-C₁₀ alkyls, m, n, p, r and s are integers being either 0 or 1, independently.

### EXAMPLES

### Example 1

### Production of Cell Samples

### Hematopoietic Stem Cells

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min). The mononuclear cell fragment was collected from the gradient and washed twice with PBS.

Umbilical cord blood cell isolation and culture. CD34 positive and negative cells as well as CD133 positive and negative cells from human umbilical cord blood were isolated using magnetic affinity cell sorting and double selection (Miltenyi Biotec, Germany) as described in Kekäräinen et al (2006, BMC Cell Biol 7:30). Washed cell pellets were frozen and stored at −70° C. prior mass spectrometric or Western Blotting analysis. For FACS analysis cells were used fresh.

### Mesenchymal Stem Cells

### Cord Blood Derived Mesenchymal Stem Cell Lines

Umbilical cord blood cell isolation and culture. From collected umbilical cord blood CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1× ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium, except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/cm². In later passages the cells were detached with trypsin/EDTA from defined area at defined time points, counted with hematocytometer and replated at density of 2000-3000 cells/cm².

### Bone Marrow Derived Mesenchymal Stem Cell Lines

Isolation and culture of bone marrow derived stem cells. Bone marrow (BM)—derived MSCs were obtained as described by Leskelä et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

### Mesenchymal Stem Cell Phenotype Determination

Both UBC and BM derived mesenchymal stem cells were phenotyped by flow cytometry (FACSCalibur, Becton Dickinson). Fluorescein isothicyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abcam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD105 and HLA-ABC.

### Adipogenic Differentiation

To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.4 μM dexamethasone (Sigma-Aldrich) and penicillin-streptomycin (Gibco) before samples were prepared for glycome analysis. The medium was changed twice a week during differentiation culture.

### Osteogenic Differentiation

To induce the osteogenic differentiation of UCB and BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

### Embryonic Stem Cells

Human embryonic stem cell lines (hESC)—Generation of the Finnish hESC lines FES 21, FES 22, FES 29, and FES 30 has been described (Mikkola et al. 2006 BMC Dev. Biol. 6:40). Briefly, two of the analysed cell lines were initially derived and cultured on mouse embryonic fibroblast (MEF) feeders, and two on human foreskin fibroblast (HFF) feeder cells. For the present studies all of the lines were transferred on HFF feeder cells and cultured in serum-free medium supplemented with Knockout serum replacement (Gibco). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium without bFGF. For further differentiation (into stage 3 differentiated cells) EB were transferred onto gelatin-coated culture dishes in media supplemented with insulin-transferrin-selenium and cultured for 10 days.

### Example 2

### Assignment of the Mass Spectrometric Glycome Signals to Disialylated Structures

### Cell Harvesting for Glycome Analysis

Mesenchymal stem cells. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times. The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

Embryonic stem cells. For glycan analysis, the cells were collected mechanically, washed, and stored frozen until the analysis. In fluorescence-assisted cell sorting (FACS) analyses 70-90% of cells from mechanically isolated hESC colonies were typically Tra 1-60 and Tra 1-81 positive. The differentiation protocol favors the development of neuroepithelial cells while not directing the differentiation into distinct terminally differentiated cell types. Stage 3 cultures consisted of a heterogenous population of cells dominated by fibroblastoid and neuronal morphologies.

Hematopoietic stem cells. Isolated and washed cell pellets were frozen and stored at −70° C. prior mass spectrometric glycan analysis.

### Glycan Isolation for Mass Spectrometric Analysis

Asparagine-linked glycans were detached from cellular glycoproteins by F. meningosepticum N-glycosidase F digestion (Calbiochem, USA) essentially as described (Nyman et al 1998 Eur. J. Biochem. 253:485). Cellular contaminations were removed by precipitating the glycans with 80-90% (v/v) aqueous acetone at −20° C. and extracting them with 60% (v/v) ice-cold methanol. The glycans were then passed in water through C₁₈ silica resin (BondElut, Varian, USA) and adsorbed to porous graphitized carbon (Carbograph, Alltech, USA). The carbon column was washed with water, then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C₁₈ silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used.

### Mass Spectrometry

MALDI-TOF mass spectrometry was performed with a Bruker Ultraflex TOF/TOF instrument (Bruker, Germany) essentially as described (Saarinen et al 1999 Eur. J. Biochem. 259:829). Relative molar abundancies of neutral and sialylated glycan components can be accurately assigned based on their relative signal intensities in the mass spectra when analyzed separately as the neutral and sialylated N-glycan fractions. Each step of the mass spectrometric analysis methods was controlled for reproducibility by mixtures of synthetic glycans or glycan mixtures extracted from human cells.

### Data Analysis

The mass spectrometric raw data was transformed into the glycan profiles by carefully removing the effect of isotopic pattern overlapping, multiple alkali metal adduct signals, products of elimination of water from the reducing oligosaccharides, and other interfering mass spectrometric signals not arising from the original glycans in the sample. The resulting glycan signals in the presented glycan profiles were normalized to 100% to allow comparison between samples.

In glycome profiles generated numerous “unusual” mass signals were managed to be assigned to di- or oligosialylated monosacchride compositions as described in Tables 1-3. Glycosidase analysis by specific sialidases and galactosidases were performed in order to increase the structural information and allow more specific assignment. For part of the structures with sensitivity to a3-sialidase of Streptococcus alternative unusual assignments were revealed. Based on the data and presence of two sialic acids and one N-acetyllactosamine unit or three sialic acids and two N-acetyllactosamine units the structures shown schematically in Tables 1-3 and in formulas of the description were obtained.

### Example 3

### FACS Analysis of Hematopoietic and Mesenchymal Stem Cells by Anti-Disialic Antibodies

### Hematopoietic Stem Cells

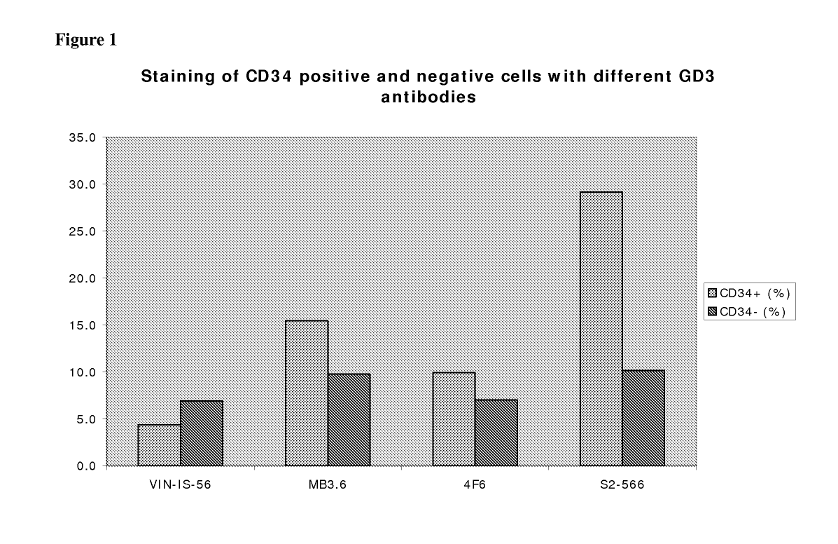

The FACS analysis of hematopoietic stem cells were performed from cord blood mononuclear cell populations using double labelling analysis with the stem cell population antibodies CD34 and CD133. FIG. 1 shows staining results of CD34 positive and negative cells with different GD3 antibodies. VIN-IS-56 was from Chemicon with (product code MAB4308), MB3.6 was from BD Pharmingen (product code 554274), 4F6 was from Covalab (product code mab0014) and S2-566 was from Seikagaku (product code 270554). The data revealed especially effective and specific labelling of the majority of CD34+ cells by antibody S2-566, known to able to recognize the preferred disialic acid epitope on proteins (Sato C. et al. J. Biol. Chem. 200, 275:15422). Antibodies MB3.6 and 4F6 have not been reported to have effective protein recognition but may have some cross reactivity as there was partially, though much lower reactivity favouring the stem cell population. Antibody VIN-IS-56 showed no preferential labelling of hematopoietic stem cells.

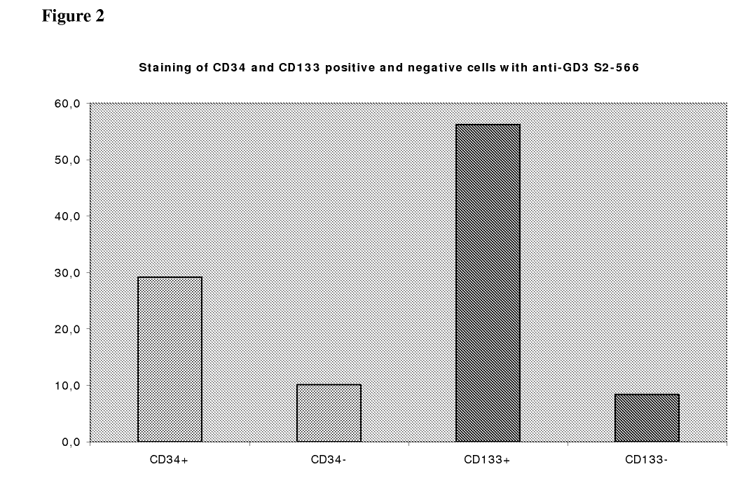

FIG. 2 shows FACS staining results of cord blood derived hematopoietic stem cells, CD34 and CD133 positive cells, and CD34 and CD133 negative cells labelled with anti-GD3 S2-566 (Seikagaku). The high staining efficiency of CD133+ cells indicates that the antibody recognized more primitive stem cell population than CD34+. The data revealed that the antibody was especially useful for recognition and isolation of hematopoietic stem cells, especially derived from cord blood. The low reactivity with the corresponding negative cells indicated that the FACS or other isolation method such as magnetic particle cell purification method using the above antibody produced highly enriched stem cell fraction. The invention is especially directed to the use of a binder recognizing the disialic acid epitope for analysis and isolation of hematopoietic stem cells. The antibody was also useful for characterization of hematopoietic stem cell populations.

### Mesenchymal Stem Cells

FIG. 3 shows FACS staining results of mesenchymal stem cells (MSC) and osteogenically (OG) as well as adipogenically (AG) differentiated cells. Bone marrow (BM) derived MSC staining is visualized in FIG. 3A and cord blood (CB) derived in FIG. 3B with different anti-disialic acid antibodies. VIN-IS-56 was from Chemicon with (product code MAB4308), MB3.6 was from BD Pharmingen (product code 554274), 4F6 was from Covalab (product code mab0014), S2-566 was from Seikagaku (product code 270554) and 4i283 was from US Biological (product code G2005-67). All GD3 antibodies labelled only part of BM derived cells with no difference regarding their cellular differentiation state. Instead there was markedly enhanced labelling of cord blood derived MSCs differentiating either into osteogenic or adipogenic direction with all gangliospecific GD3 antibodies tested. No clear difference was observed with the “protein/lactosamine disialic acid” antibody S2-566 and other “ganglio disialic acid” GD3 antibodies. MB3.6 is however considered to have somewhat similar specificity as S2-566 and it is considered as less preferred alternative for recognition of especially differentiated cord blood mesenchymal stem cells as in FIG. 3B. The invention further revealed completely different specificity of O-acetyl GD3 derived sialic acid labelling antibody (4i283, US Biological). No binding to stem cells or to cells differentiated thereof was observed.

FIG. 4 shows more specific FACS analysis of mesenchymal stem cells (MSC) and osteogenically differentiated (OG) and adipogenically (AG) differentiated cells from bone marrow (BM) and cord blood (CB) with antibody S2-566 (Seikagaku).

### Example 4

### Immunoblotting of Hematopoietic Stem Cell Lysate with Anti-Disialic Acid Antibody

CD34 positive and negative cells from human umbilical cord blood were isolated using magnetic affinity cell sorting as described in Example 1. Cell pellets were frozen and stored at −70° C. Thawed cells were lysed in 1% Triton X-100, 10 mM sodium phosphate, 300 mM NaCl, pH 7.4 with protease inhibitors at 60×10⁶ cells/ml for 15 minutes on ice. Lysates from multiple umbilical cord blood units were pooled together. The pooled lysate was cleared by centrifugation at 13 000 rpm for 10 min.

Cell lysate of 27 μg total protein (determined by Bradford) per lane was run on 10% SDS-PAGE gel which was further blotted onto PVDF membrane. The membrane was blocked with 1% BSA in PBS containing 0,1% Tween-20. The membrane was incubated with primary antibody S2-566 (Seikagaku) (1 μg/ml in PBS, 0,1% Tween-20, 0,1% BSA) overnight at +4° C. After washing with PBS containing 0,05% Tween-20, the membrane was incubated with peroxidise-conjugated goat anti-mouse IgG+IgM (1:5000 dilution; ThermoScientific). Detection was performed using Amersham ECL Western Blotting Detection Reagents (GE Healthcare).

FIG. 6 reveals specific binding of anti-disialic acid S2-566 (Seikagaku) to a protein of CD34+ hematopoietic stem cell lysate. The particular protein has an approximate molecular weight of 45 kDa estimated from molecular weight markers visualized in the gel. In corresponding differentiated CD34− cells no staining could be visualized. In control experiment known glycolipid antibody VIN-IS-56 did not show any strong or specific binding to any glycoprotein in the hematopoietic stem cell lysate blot.

### Tables

TABLE 1
Presence of oligosialylated lactosamine structures in N-glycomes of CD133+
hematopoietic stem cells.

| | CD133+ | CD133- | |
|---|---|---|---|
| m/z | % | % | |
| 1856 | 0.87 | 0 | 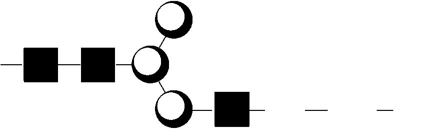 |
| 2294 | 1.65 | 0 | 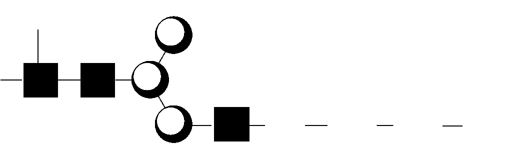 |

m/z indicates mass to charge ratio, % indicates the relative amount of glycan type from total glycome of the cells.
Preferred structure types are indicated in color coded structures, square (blue/dark) is GlcNAc, circle Man (green), yellow Gal (light), NeuNAc is indicated by diamonds.

TABLE 2
Presence of oligosialylated structures in N-glycomes of human embryomal stem cells.

| | St1 | St2 | St3 | mEF | |
|---|---|---|---|---|---|
| m/z | % | % | % | % | |
| 1840 | 0.2 | 0.4 | 0.6 | 0 | 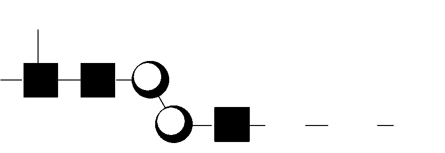 |
| 2002 | 0.3 | 1.1 | 0.9 | 0.2 | 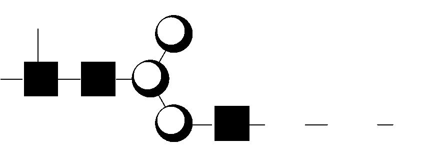 |
| 2408 | 1.1 | 0.3 | 0 | 0 | 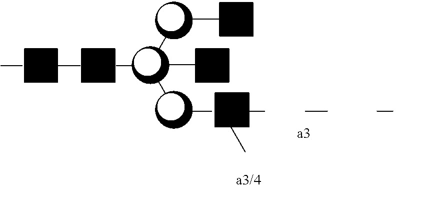 |
| 2528 | 0.2 | 0.1 | 0 | 0 | 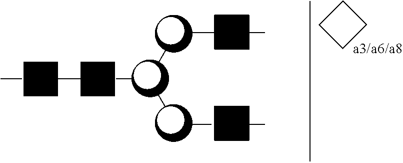 |
| 2544 | 0.2 | 0 | 0 | 0 | 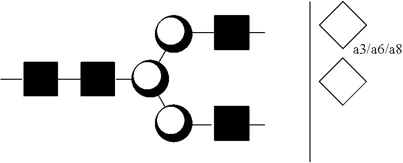 |

m/z indicates mass to charge ratio, % indicates the relative amount of glycan type from total glycome of the cells.
Preferred structure types are indicated in color coded structures, square (blue/dark) is GlcNAc, circle Man (green), yellow Gal (light), NeuNAc is indicated by diamond (mangenta), NeuGc by diamond (light blue); St1 is stage 1 (non-differentiated), St2 is stage 2 differentiated, St3 is stage 3 differentiated and mEF is control mouse feeder cells. The structures correspond to monosaccharide compositions S2H3N3F1, S2H4N3F1, S2H4N5F1, and biantennary type structures α3/α6/α8-linked sialic acids S2G1H5N4, and S1G2H5N4.

TABLE 3
Relative amounts of glycan types in different types of mesenchymal stem cells and cell populations derived thereof.

| | BM MSC | BM MSC | BM MSC | osteo- | AB | | |
|---|---|---|---|---|---|---|---|
| | (Abserum) | (FCS) I | (FCS) II | geeniset | serum | FCS | |
| m/z | % | % | % | % | % | % | |
| 1694 | 0 | 0 | 0.06 | 0 | 0 | 0 | 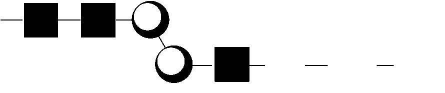 |
| 1840 | 0.26 | 0.19 | 0.13 | 0 | 0 | 0 | 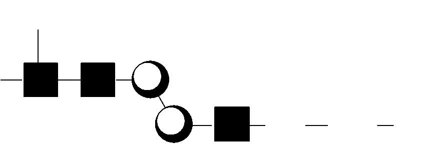 |
| 1856 | 0 | 0 | 0 | 1.37 | 0 | 0 | 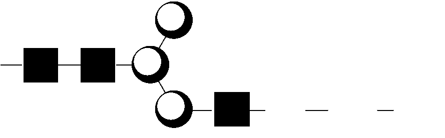 |
| 2002 | 0.32 | 0.32 | 0.34 | 0.18 | 0 | 0 | 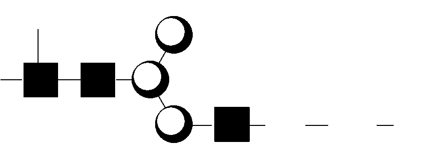 |

m/z indicates mass to charge ratio, % indicates the relative amount of glycan type from total glycome of the cells.
Example structure types are indicated in color coded structures, square (blue/dark) is GlcNAc, circle Man (green), Gal (light/ yellow), NeuNAc is indicated by diamonds (mangenta). BM MSC indicates bone marrow mesenchymal stem cells (two representative data set shown). “osteogeeniset” indicates bone marrow mesenchymal stem cells differentiated to osteogenic cells.

1-31. (canceled)

32. Method for analyzing status of human stem cells or differentiated cells derived therefrom by analyzing the amount of or presence of a structure in a cell sample containing human cells, said structure comprising at least two sialic acid residues per
a. one N-acetyllactosamine, wherein the human cells are mesenchymal, embryonal or hematopoietic stem cells and/or
b. one lactose residue of GD3 ganglioside, wherein the human cells are differentiated mesenchymal cells, or hematopoietic stem cells;
with the proviso that the sialic acids form structure NeuXα8NeuXα3Gal, wherein X is Ac or Gc or —OAc,
or non-linear disialylated N-acetyllactosamines comprising one sialic acid on position 3 of Gal and another one on position 6 of GlcNAc.

33. Method according to claim 32, wherein NeuNAcα8NeuNAcα3Gal-epitopes are recognized and the disialic acid epitope is presented as
a1) NeuNAcα8NeuNAcα3Gal on a protein and/or N-acetyllactosamine epitope, for analysis of differentiated mesenchymal cells, or hematopoietic stem cells, and/or
a2) NeuNAcα8NeuNAcα3Gal on ganglioseries ganglioside GD3 or OAcGD3, for analysis of hematopoietic stem cells or for analysis of differentiated mesenchymal cells in combination with structure according to point a. in claim 32.

34. Method according to claim 33, wherein NeuNAcα8NeuNAcα3Gal-epitopes are recognized and the disialic acid epitope is presented as NeuNAcα8NeuNAcα3Gal on a protein and/or N-acetyllactosamine epitope and the cells are hematopoietic stem cells or differentiated mesenchymal cells.

35. The method according to claim 32, wherein the analysis is performed by using mass spectrometry or by using specific binding agent/binder recognizing the epitope.

36. The method according to claim 35, wherein the binder is S2-566 antibody.

37. The method according to claim 35, wherein binders for N-acetyllactosamine and/or protein linked structure, and for gangliosides as in claim 33 are used together.

38. The method according to claim 35, wherein the antibodies are used to analyze differentiated mesenchymal cells

39. The method according to claim 32, wherein the structure is a “non-linear disialylated” N-acetyllactosamine comprising one sialic acid on position 3 of Gal and another one on position 6 of GlcNAc, i.e. NeuXα3Galβ3(NeuXα6)GlcNAc, wherein X is Ac or Gc.

40. Method for selection or production of antibodies for analysis, including purification, of differentiated mesenchymal cells, hematopoietic stem cells and cells directly differentiated thereof, comprising a step of screening antibodies recognizing the structure as defined in claim 32, wherein the analysis is performed according to claim 32.

41. The method according to claim 32 wherein the structure is an N-glycan having a preferred N-monosaccharide composition according to Formula C
SₖHₙNₚF_{q}
wherein
k is integer from 2 to 3,
n is integer 3 or 5,
p is integer 3 or 4,
q is integer being 0 or 1,
with the provision that when n is 3, then p is 3 or 4, or when n is 5 then p is 4 and k is 3 and
S is Neu5Ac and/or Neu5Gc, H is hexose selected from group D-Man or D-Gal, N is N-D-acetylhexosamine, preferably GlcNAc or GalNAc, more preferably GlcNAc, and F is L-fucose.

42. The method according to claim 41, wherein the structure has composition S2G1H5N4, S1G2H5N4, or S2H4N5F1

43. The method according to claim 32, wherein the structure is a N-glycan according to Formula OS1
(NeuAcα)ₘGalβ(Fucα3/4)ₙ₁GlcNAcβ2Manα3([Manα6]ₙ₂)Manβ4GlcNAcβ4(Fucα6)ₙ₃GlcNAc, wherein n1, n2 and n3 integers 0 or 1, with the provision, that when n1 is 0 then n3 is 1 and when n1 is 1 then n3 is 0 or both n1 and n3 are 0 and wherein m is integer 2 or 3, and wherein sialic acid residues are α3-linked to Gal and α8-linked to each other.

44. The method according to claim 32, wherein the structure is a N-glycan according to the Formula
(NeuXα)ₘ₁GalβGlcNAcβ2Manα3([NeuXα]ₘ₂GalβGlcNAcβ2Manα6)Manβ4GlcNAcβ4G lcNAc,
wherein X is either Gc or Ac, with the provision that there is at least one Gc or Ac in the molecule and that there can be both Gc and Ac in disialic acid epitopes and m1 is 2 and m2 is 1, or
m2 is 2 and m1 is 1, and sialic acid residues are α3-linked to Gal and α8-linked to each other; the Gal residues are either β3 and/or β4 linked.

45. The method according to claim 32, wherein the structure is a N-glycan according to Formula
NeuXα3Galβ3(NeuXα6)GlcNAcβ2Manα3 (NeuXαGalβ3 GlcNAcβ2Manα6)Manβ4GlcN Acβ4GlcNAc
and/or other branch isomer
NeuXαGalβ3GlcNAcβ2Manα3/6(NeuXα3Galβ3(NeuXα6)GlcNAcβ2Manα6/3)Manβ4G lcNAcβ4GlcNAc.

46. The method according to claim 32, wherein differentiation of cells or differences in cell types or cell contamination is analyzed.

47. The method according to claim 32, wherein said method is used for isolation or purification of mesenchymal, embryonal or hematopoietic stem cells or differentiated mesenchymal cells.

48. An isolated or purified cell sample of mesenchymal, embryonal or hematopoietic stem cells or differentiated mesenchymal cells obtained by the method according to claim 47.

49. The cell sample according to claim 48, wherein the cell sample is isolated by antibody S2-566 and contains a hematopoietic stem cell population.
